(19) United States
(12) Patent Application Publication
(10) Pub. No.: US 2004/0191856 A1
(43) Pub. Date: Sep. 30, 2004

(54) Online chemical reaction device and analysis system

(75) Inventors: Atsumu Hirabayashi, Kodaira (JP); Yoshinobu Kohara, Kokubunji (JP); Kazunori Okano, Shiki (JP)

Correspondence Address: Stanley P. Fisher, Reed Smith LLP, Suite 1400, 3110 Fairview Park Drive, Falls Church, VA, 22042-4503, US

(73) Assignee: Hitachi, Ltd.

(21) Appl. No.: 10/750,886

(22) Filed: Jan. 5, 2004

(30) Foreign Application Priority Data
Mar. 24, 2003 (JP) 2003-079346

(51) Int. Cl. C12P 1/00

(52) U.S. Cl. 435/41

(57) ABSTRACT

In the batch process of enzyme-catalyzed reactions, a sample loss cannot be negligible, but devices according to the prior art aiming at reduction in sample loss cannot help consuming a long period of time in the reaction process. In the present invention, a chemical substance is fixed and a buffer solution is filled in a round-shaped reaction element. A given volume of air is sucked in through an air inlet and a sample solution is sucked in. In addition, a given volume of air is sucked in. Air and the sample solution, is transported sequentially into the reaction element in that order. The reaction element is filled with the sample solution, which is placed between two air layers to prevent it from mixing with the buffer solution. The sample solution between two air layers reciprocates at a given velocity of flow in the reaction element, accelerating high efficiency chemical reactions.

### PRIORITY CLAIM

This application claims priority under 35 U.S.C. §119 to Japanese patent application P-2003-079346 filed Mar. 24, 2003, the entire disclosure of which is hereby incorporated herein by reference.

### FIELD OF THE INVENTION

The present invention relates to the chemical reactions of minute samples and in particular to the reactions of proteins, peptides, sugar chains, and genes. In addition, the present invention relates to an analysis system for the chemical reactions of minute samples.

### BACKGROUND OF THE INVENTION

Some techniques have been developed for improving the activities of enzymes in enzyme-catalyzed reactions and minimizing sample loss due to the introduction of an online system. For example,a patent document 1, JP-A No. 313196/1994, describes the method for enzyme-catalyzed reaction using chitosan beads (0.5 mm to 3 mm in diameter) , on which an enzyme has been fixed (“patent document 1”). This technique, which involves the steps of adding enzyme-fixed beads into a sample solution and shaking the solution, accelerates the chemical reaction by dispersing the enzymes fixed on the beads in the sample solution. Although the technique with enzyme fixed on the beads can improve substantially the enzyme activity, it requires about 1 to 50 hours of reaction time. For example, a patent document 2, JP-A No. 196897/1999, describes an online enzyme reaction technique for minimizing sample loss (“patent document 2”). This technique involves the steps for filling enzyme-fixing catalyst support gel into columns and transporting the sample solution onto the column via a pump. Although the method enables an analysis system to be automated, it is estimated to take about several hours of reaction time. Being not the case of enzyme-catalyzed reaction, a probe array (bead array) , in which particles such as beads with a chemical, for example a probe, bonded on their surfaces, are arrayed, is described in, for example Patent Document 3, JP-A No. 243997/1999. In this example, the dispensing the sample solution into the probe array allows for the specific bonding of a sample substance to a sample, which can be spectrometrically detected. Different chemicals may be bonded to the individual molecules of the sample substance, but no information on how to optimize its reaction efficiency has been provided so far.

To accelerate online enzyme-catalyzed reactions, it is effective to increase the surface areas of the fixed enzyme beads. For example, a non-patent document 1, Analytical Chemistry Vol. 72(2000) , pp. 286-293, describes a technique for forming 32 fine flow channels (50 μm in width, 250 μm in depth, 11 mm in length) on a silicone substrate to fix the enzyme on them (“non-patient document 1”). This technique allowing for larger surface areas of the beads, on which the enzyme is fixed, reduces the enzyme-catalyzed reaction time. Note that it has such a disadvantage that the sample is introduced into the fine flow channels at a given rate, which causes water pressure to extremely rise, affecting reaction efficiency. Besides, a technique for forming porous monolithic columns in a capillary and fixing the enzyme on the formed monolithic columns (enzyme-fixed monolithic columns) is described in a non-patent document 2, Analytical Chemistry Vol. 74(2002), pp. 4081-4088. With this technique, the surface areas, on which the enzyme is fixed, can be made far larger, which reduces the reaction time, achieving higher throughput. In addition, the porous property of the monolithic columns allows for sample dispensing at a relatively low pressure. On the other hand, the technique has a disadvantage of. the manufacturing process of monolithic columns being troublesome even at a higher cost.

Conventionally, the enzyme-catalyzed reactions in sample solutions have often proceeded in the vessels using a batch method but in such a manner of batch process, sample loss cannot be negligible. Moreover, another problem of reduction in enzyme activity has risen. For that reason, the batch system tends to have a disadvantage in the chemical reaction process of minute samples because of samples being of minute amount.

In contrast, any conventional technique aiming at reduction in sample loss takes as a long time as several hours to several tens hours until the reaction finishes. With respect to the reaction method using beads, no information on the method for optimizing reaction efficiency has been provided.

### SUMMARY OF THE INVENTION

To solve these problems, any chemical reaction method is required, which allows minute samples to be chemically processed with low loss, and the device for implementing it.

To achieve the chemical process of the samples in a shorter period of time and with less sample loss, the chemical reaction method, which allows for a increasing number of times molecules chemically cross-react, and the device for implementing it are essential.

According to one aspect of the present invention, sample flow channels for dispensing supports, or supports, with biological molecules fixed on their surfaces are formed. The sample is introduced around the area where the supports are set in the sample flow channels with the structure avoiding the diffusion of the sample. That is, there is the separation region between the sample and the solutions which is used for transporting the samples. The sample can be introduced into the sample flow channels using other solutions and/or air gaps. One of the way for the sample introduction is the introduction of a first solution, a first air layer, the sample, a second air layer and a second solution in turn. Then, the first solution and the first air layer and the sample and the second air layer and the second solution are moved so that the sample may move relative to the supports, initiating the reaction between the biological molecules and the substance contained the sample solution.

An enzyme may be used for the biological molecules, wherein the above-mentioned reaction is an enzyme-catalyzed digestion of the sample substance.

The first air layer and the second air layer may be structured so that one end of the sample flow channel is connected to a first pipe and the other end of the sample flow channel is connected to a second pipe, respectively, wherein the first air layer and the second air layer are disposed at the first pipe and the second pipe, respectively.

The volume of the sample solution to be introduced into the sample flow channel may be any of a range from more than 0.1 μL (including) to less than 100 μL (including) and may be more than the volume obtained by subtracting the volume of micro particles from the inner capacity of the sample flow channel.

Placing the first and second air layers between the first solution and the sample and between the second solution and the sample prevent, respectively, prevents the first and the second solutions and the sample to be mixed.

According to the chemical reaction method above mentioned, the supports and the sample flow channels may be beads and capillaries, respectively and the latter may be flow channels formed on a reaction vessel as with a structure formed in the reaction vessel.

A chemical reaction device and an analysis system using this device include capillaries containing micro particles and the first and second pumps the rates of the fluid flowing in the capillaries. In addition, they have a first pipe, which connect to one end of the capillary and to a first pump, a second pipe, which connects to the other and of the capillary and to a second pipe, and an air blowing tube, a solution feeding tube, and a sample feeding tube, which connect to either the first pipe or the second pipe. The air blowing tube supplies air between the fed solution and the fed sample.

The above-mentioned sample may reciprocate.

In addition, they may also have a connecting valve for selectively connecting the air blowing tube, the solution feeding tube, and the sample feeding tube to at least either the first pipe or the second pipe.

Alternatively, they may have not only the first connecting valve for selectively connecting at least any of the air blowing tube, the solution feeding tube, and the sample feeding tube to the first pipe but also a second connecting valve for selectively connecting at least any of the air blowing tube, the solution feeding tube, and the sample feeding tube to the second pipe.

The above-mentioned chemical reaction device may be used as any of a stand-alone system, a serial connection of a plurality of devices to perform sequentially a series of chemical reactions, and a parallel connection of a plurality of devices to perform simultaneously chemical reactions. Alternatively, the device may be an online chemical reaction device or a mass analysis system.

### BRIEF DESCRIPTION OF THE DRAWINGS

FIG. 1 is a structural view showing a chemical reaction device according to one embodiment of the present invention;

FIG. 2 is a schematic view of a reaction part 1 of the chemical reaction device according to one embodiment of the present invention;

FIG. 3 is a schematic view of a protocol of a transported liquid for the chemical reaction device according to one embodiment of the present invention;

FIG. 4 is an operation sequence of the chemical reaction device in conformity to the transported liquid protocol according to one embodiment of the present invention;

FIG. 5 is a structural view of the chemical reaction device according to one embodiment of the present invention;

FIG. 6 is an example of enzyme-catalyzed digestion of the sample substance in the chemical reaction device according to one embodiment of the present invention;

FIG. 7 is an example of enzyme-catalyzed digestion of proteins according to one embodiment of the present invention;

FIG. 8 is a schematic view of the chemical reaction device,, in which structures according to one embodiment of the present invention is disposed at the flow channel;

FIG. 9 is a structural view of an analysis system (shotgun analysis system), which has incorporated a protocol for analyzing proteins using a mass spectrometer and a chemical reaction device according to one embodiment of the present invention;

FIG. 10 is a detail view of a 2D-HPLC element;

FIG. 11 is a structural view of an analysis system, which has incorporated a protocol for analyzing a sugar chain structure (a sugar chain sequence) using a mass spectrometer and a chemical reaction device according to one embodiment of the present invention; and

FIG. 12 is a structural view of an analysis system, in which plurality of chemical reaction devices operate in parallel.

### DETAILED DESCRIPTION OF THE PREFERED EMBODIMENTS

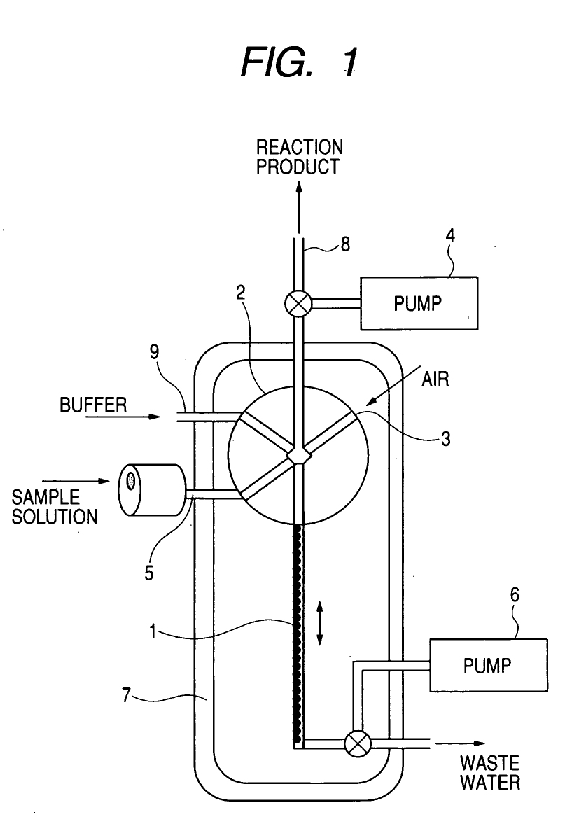

FIG. 1 shows a structural view of a chemical reaction device according to one embodiment of the present invention. A reaction element 1 shaped into a tube has a chemical substance fixed. A buffer solution is filled in a valve 2 and the reaction element 1. First of all, a given volume of air is sucked from an air inlet 3 to a valve 2 by a second pump 4. Next, a sample solution is sucked in from the sample inlet 5 to the valve 2 by the second pump 4. Then, a given volume of air is sucked in from the air inlet 3 to the valve 2 by the second pump 4. Further then, the sucked air, sample solution, and air are sequentially transported to the reaction element 1 by a first pump 6 and the second pump 4. Accordingly, the reaction element 1 is filled with the sample solution and both boundaries of the reaction element 1 are blocked by the sucked-in air to prevent the sample solution from mixing with the buffer solution. The temperature of the reaction element 1 is controlled to for example about 37° C. in a constant temperature element 7. Only a certain volume of the sample solution reciprocates in the reaction element 1 at a given velocity of flow by means of the first pump 6 and the second pump 4. After the sample solution reciprocates for a given period of time, a chemical reaction finishes in the reaction element 1. The sample (a reaction product), on which the chemical reaction was made, is discharged from an outlet 8 through the valve 2. Then, the reaction element 1 and the valve 2 are cleaned with the buffer solution introduced through a buffer inlet 9 for cleaning. Moreover, during the storage period, in which no sample solution is introduced, the temperature of the reaction element 1 is dropped down to about 4° C., preventing the fixed chemical substance from degenerating.

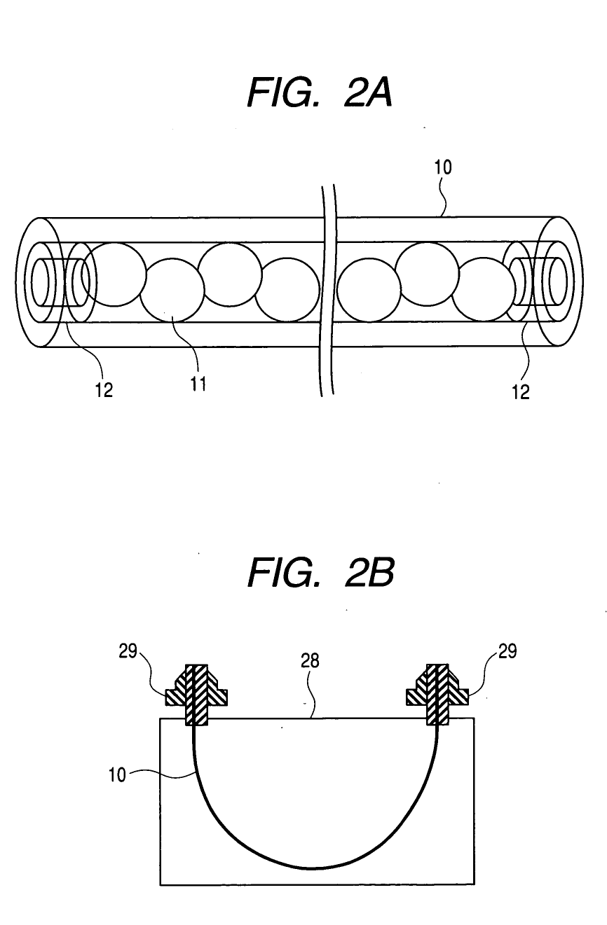

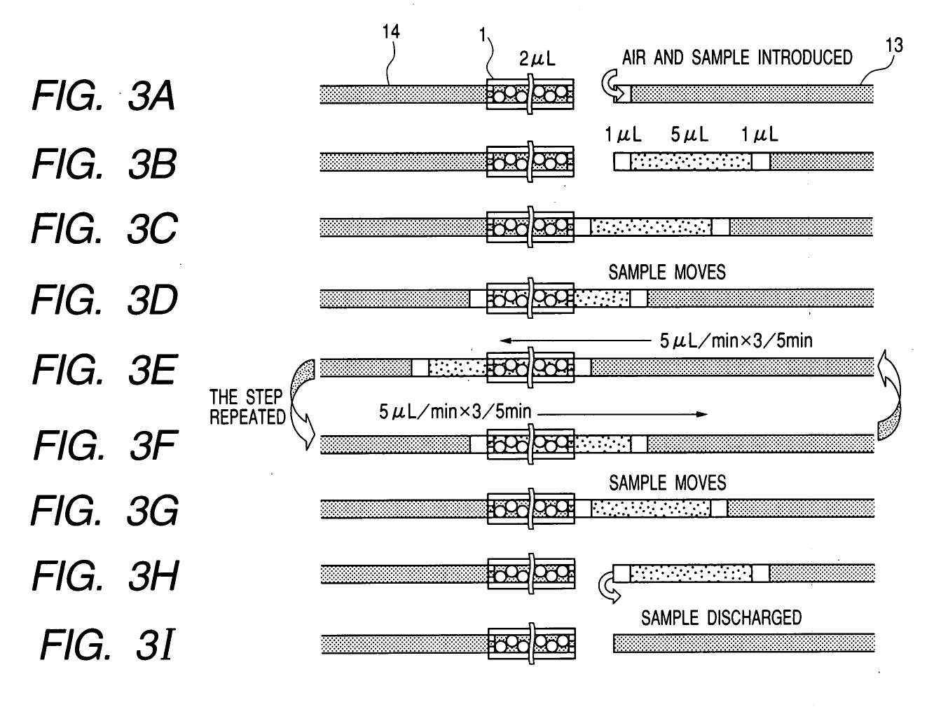

The structure of the reaction element 1 of a chemical reaction device according to one embodiment of the present invention is shown in FIG. 2A. Only about 2800 glass beads 11 (103 μm in diameter) with enzyme (trypsin) molecules fixed on their surfaces, are introduced into a capillary 10 with a length of 200 mm (50 μmm in internal diameter, 360 μm in external diameter). The capillary 10 have another capillaries 12 (50 μm in internal diameter, 150 μm in external diameter, 5 mm in length) inserted at its both ends to fix the glass beads inside it. It is useful that a coating film is applied to the inner surfaces of the capillaries 10 and 12 to prevent the sample from being absorbed and alternatively, the same chemical substance as with the glass beads 11 may be fixed on them. The capacity of the reaction element having such a structure is about 2 μL.

As an example, the method for producing the trypsin-fixed glass beads is described. The following procedure can be used to fix trypsin molecules on the glass beads with surfaces qualified with amino groups.

1. A step for substituting the amino groups fixed on the bead surfaces with carboxyl groups: Putamino-group qualified glass beads (100 mg) into a polypropylene tube (2 mL of capacity) and add 500 μL of 480 mM succinic anhydride solution (solvent: 1-methyl-2-pyrrolidone) in it.

2. Stir the succinic anhydride solution and the beads together in the tube at 50° C. for 60 minutes.

3. Add 500 μL of 0.1 M boric acid buffer (pH8.0) in the tube and leave it at 20° C. for 10 minutes.

4. Clean the beads in the tube with 1 mL of pure water. Repeat this cleaning step six times.

5. A step for activating carboxyl groups: Clean the beads with a mixture solution of 20 mM N-hydroxy succinimide and 0.1 M N-ethyl-N′-3-dimethylaminopropylcarbodimide (1 mL, solvent: 0.1 M boric acid buffer (pH6.2)) once.

6. Add a mixture solution of 20 mM N-hydroxy succinimide and 0.1M N-ethyl-N′-3-dimethylaminopropylcargbodimide (1 mL, solvent: 0.1 M boric acid buffer (pH6.2)) in the beads. Leave the beads in the tube on ice for 30 minutes (while stirring them occasionally) and then collect only the beads.

7. Clean the beads with 200 μL of 0.1 M boric acid buffer (pH6.2)

8. A step for fixing Trypsin: Dissolves 40 mg of trypsin into 800 μL of 0.1 M boric acid buffer (pH6.2) and add it in the beads. Leave it at 4° C. overnight (16 hours).

9. Clean the beads with 2 mL of 10 mM Tris-HCl solution (pH8.0). Repeat this cleaning step six times.

10. Immerse the beads in the 10 mM Tris-HCl solution (pH8.0) and then store them at 4° C.

The application of the method for fixing a chemical substance mentioned above is not always limited only to the fixation of trypsin. The resulting enzyme-fixed glass beads 11 can be filled in the capillary 10 as shown in FIG. 2A by sucking them together with the buffer solution by means of any means, for example a pump, used in the capillary 10. To observe the state of bead filling, it is desired that the material for the beads is transparent to some degree. This type of enzyme-fixed glass beads 11, when being dried, tends to deteriorate the activity of enzyme on them. To address this problem, it is desired that the buffer solution is filled in the manufactured reaction element 1, attach any cover, for example a lid, to it to prevent the enzyme-fixed glass beads 11 from being dried, and store them at 4° C. This process enables the activity of the enzyme in the reaction element 1 to be kept even when the reaction element 1 is used repeatedly.

A protocol for transporting a liquid in a chemical reaction device according to one embodiment of the present invention is schematically shown in FIG. 3. In the figure, a second pump 4 on the left side and a first pump 6 on the right side are connected to capillaries 14 and 16, respectively. It is assumed that the capacity of the reaction element 1 is 2 μL and the volume of a sample is 5 μL. Any of liquids such as 10 mM Tris-HCl solution (pH8.0) has been filled in the reaction element 1, the capillary 13, and another capillary 14 but initially the reaction element 1 and the capillary 13 are not connected. (a) 1 μL of air is sucked into the open end of the capillary 13 by a second pump 4 and then (b) 5 μL of sample and additional 1 μL of air are sucked into the capillary 13 by the pump 4. (c) With the sample solution fixed between the air layers, the open end of the capillary 13 is the reaction element 1. (d) The sample solution moves to the reaction element 1 by the second pump 4 and a first pump 6 and stops when the left air layer is introduced into the capillary 14. At that time, the remaining sample volume in the capillary 13 is 3 μL. (e) The sample moves toward the left side at a flow rate of 5 μL/minute by the second pump 4 and the first pump 6, which stop when the right air layer moves to the fixed position in the capillary 13 with the sample fixed in the reaction element 1. It takes 0.6 minutes to introduce the remaining sample (3 μL) in the capillary 13 to the reaction element 1. (f) The sample moves toward the right side at a flow rate of 5 μL/minute by the second pump 4 and the first pump 6, which stop when the left air layer moves to the fixed position in the capillary 14 with the sample fixed in the reaction element 1. It takes 0.6 minutes to introduce the remaining sample (3 μL) in the capillary 14 to the reaction element 1. By repeating steps (e) and (f) for given hour (given times), the chemical reaction is accelerated. (g) The sample solution (reaction product) fixed between the air layers moves to the capillary 13 and goes into the same state as that in the step (c). (h) The reaction element 1 and the capillary 13 are disconnected and the sample solution (reaction product) is taken out. (i) The capillary 13 is filled with the buffer solution and returns to its initial state. The movement of the sample solution fixed between the air layers to the capillaries prevents the solution from mixing with any of liquids such as a Tris-HCl solution. Note that in the repetitive steps (e) and (f) , the sample solution reciprocates in the reaction element 1, but no air is desirably blown into the reaction element 1. That is because if air is blown into the reaction element more than one time, the air for preventing the sample solution and the buffer solution from mixing together is formed into fine air bubbles, which may dilute the sample solution with the buffer solution. If the volume of the sample is not larger the capacity of the reaction element 1, the sample solution may mix with the buffer solution at a high provability in the repetitive reaction process of the sample solution.

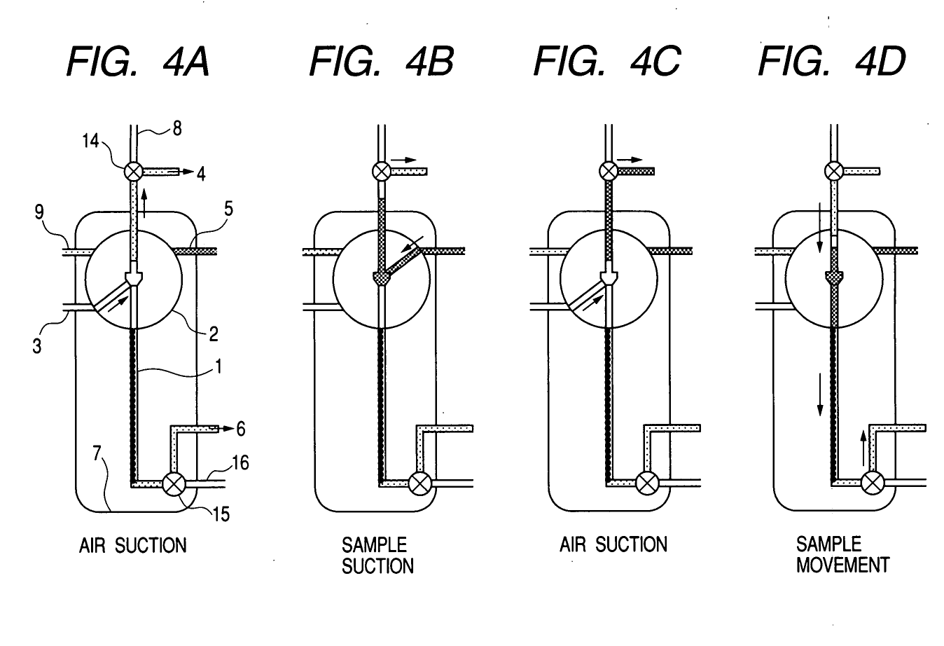

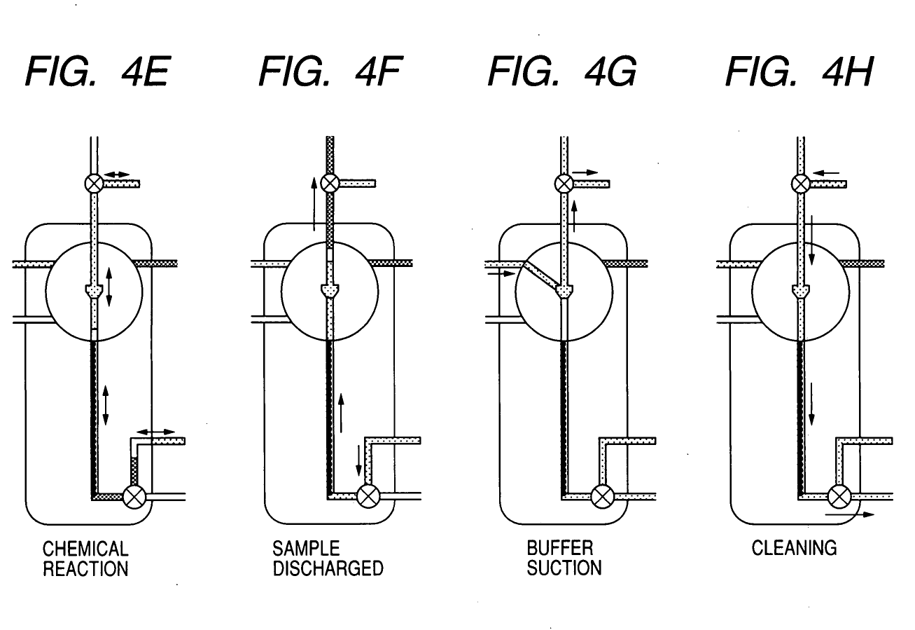

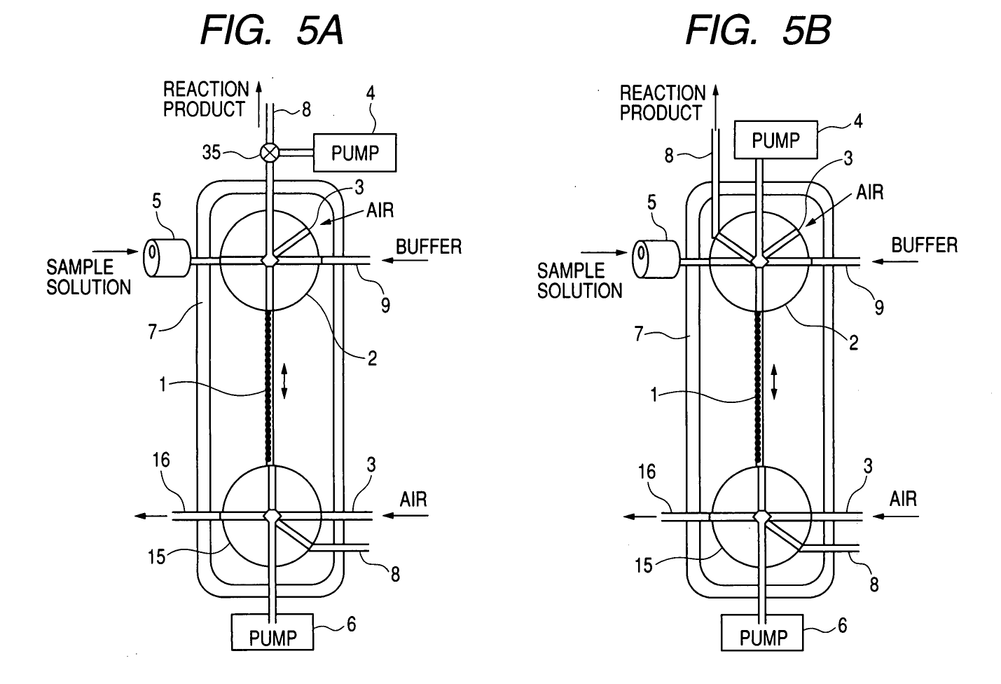

FIG. 4 shows the sequence, in which the chemical reaction device according to one embodiment of the present invention operates in conformity to the protocol for transporting a liquid. The chemical reaction device is composed of a reaction element 1, a valve 2, an air inlet 3, a sample inlet 5, a buffer inlet 9, a constant temperature element 7, a first pump 6, a second pump 4, and an outlet 8. Before the reaction is initiated, the reaction element 1 has been filled with the buffer solution and the tubing connecting to the first pump 6 and the second pump 4 have been also filled with any of liquids such as the buffer solution. The reaction element 1 is controlled to the predetermined temperature by the constant temperature element 7 composed of, for example a Peltier device. (a) The valve 2 connects the second pump 4 and the air inlet 3 and a given volume of air is blown toward tubing connected to the second pump through a valve 14 by the second pump 4. (b) Next, the valve 2 rotates twice to connect the sample inlet 5 and the second pump 4, from which a given volume of sample is introducing toward the tubing connected to the second pump 4 through the valve 2 by the second pump 4. (c) Again, the valve 2 rotates twice to connect the air outlet 3 and the second pump 4, through which a given volume of air is blown toward the tubing connected to the second pump 4 by the second pump 4. This state is shown in FIG. 3C. (d) The valve further rotates twice to connect the air/sample/air structure introduced toward the tubing connected to the second pump 4 through the valve to the inlet of the reaction element 1. The sample is moved toward the first pump 6 by the first pump 1 and the second pump 4 until the reaction element 1 is fully filled. (e) Only for a given hour, the sample reciprocates in the reaction element 1. In the case of enzyme-catalyzed digestion of protein using trypsin, the protein undergoes peptide bond in about 10 minutes. This reaction time can be predetermined depending on the type of a chemical substance fixed in the reaction element or the type of a sample. (f) The valve 14 connects the valve 2 and the outlet 8. The sample discharged from the reaction element 1 by the first pump 6 moves toward the outlet 8 and then discharged into the outside of the device. (g) The valve 14 operates to connect the tubing toward the second pump 14 and the valve 2. Then, the valve 2 rotates to connect the buffer inlet 9 and the tubing toward the second pump 4. Further then, a new buffer solution is introduced from the buffer inlet 9. (h) The valve 2 rotates to connect the tubing connected to the second pump 4 to the reaction element 1 and a new buffer solution is introduced into the reaction element 1. The new buffer solution cleans reciprocates in the reaction element 1 to clean up the reaction element 1. Then, a valve 15 operates to connect an outlet 16 and the reaction element 1 and then the buffer solution is discharged from the outlet 16 by the second pump 14. To continue the reaction process, the procedure returns to the <air suction> step mentioned above. On the other hand, to end the reaction process, the constant temperature element drops the temperature of the reaction element 1 to 4° C., preventing any deterioration in function of the reaction element 1. The reaction element 1 can be repeatedly used but must be replaced with a new one when its function deteriorates. As shown in FIG. 2B, by fixing the capillary 10 in a cell in a vessel 28 made of any of materials with a high heat conduction coefficient such as aluminum so that fittings 29 may be attached to the both ends of the capillary 10, the reaction element 1 can be easily replaced with a new one. In the example shown in FIG. 4, the air inlet 3 is connected to the valve 2 and alternatively, it may be connected to a valve 15 as shown in FIG. 5A. In such a structure, the sample solution and the buffer solution can be easily prevented from mixing together because no air is introduced into the reaction element 1. Besides, it is no problem that the outlet 8 is connected to the valve 15. In this case, a reaction product may be taken out from the outlet 8 connected to the valve 15. In the case that, for example the reaction element has a sufficiently high reaction efficiency, the reaction can be ended only by passing the sample through the reaction element once. For that reason, the reciprocating steps shown in FIG. 3E and FIG. 3F are not always necessary in this case. As shown in FIG. 5B, the pump 4 and the outlet are connected directly to the valve 2 with no valve 35. In this case, to discharge the sample solution, the sample solution is moved toward the pump 4 temporarily and then moved to the outlet 8 through the valve 2. Because of no valve 35, the structure of the device is further simplified, saving more manufacturing cost.

The condition for transporting the sample solution in the reaction element 1 is strongly related to its chemical reaction efficiency. For example, if a flow rate (velocity of flow) is considerably low, the solution forms a laminar flow. In such a case, a component of kinetic energy is generated perpendicular to the flow of the sample molecules by thermal diffusion, which governs the collisions of chemical substance particles against a fixed wall surfaces. In the collision process, the chemical reaction proceeds at a certain probability. In the case of the sample molecules being protein molecules, -the diffusion velocity is about 10 μm/second and the chemical reaction occurs only on the sample molecules in the vicinity of the wall surfaces, while it does not occur on a greater part of molecules moving at the center of the flow because it takes more time for the molecules to move to the vicinity of the wall surfaces. This means that it requires sufficient time to cause the chemical reaction on all the sample molecules. On the other hand, if the flow rate (velocity of flow) is considerably high, the liquid forms a turbulent flow. In this case, turbulent diffusion substantially causes all the sample molecules to easily collide against the wall surfaces, improving the whole reaction efficiency. The transient flow, which causes a partial but not a whole turbulent flow, is also effective because chemical reaction efficiency becomes higher than that as with the turbulent flow.

In general, it is known that when the resistance coefficient C against a round tube is proportional to the minus first power of a Reynolds number, Re, a laminar flow is formed. On the other hand, a turbulent flow indicates an intermediate dependency, for example the resistance coefficient C is proportional to the minus zero^{th} power of a Reynolds number, Re, while in the case of a flow in the transient flow zone, in which a partial turbulent flow is formed, it is proportional to the minus one-half power of a Reynolds number, Re. Turbulent diffusion is effective when the resistance coefficient C is any in a range from the zero^{th} power of a Reynolds number, Re, to the minus first power of a Reynolds number, Re. The resistance coefficient C is proportional to the minus second power of a flow rate Q and to the first power of a back pressure ΔP. The Reynolds number Re is proportional to the flow rate Q. Thus, it is concluded that turbulent diffusion is effective when the condition described below is met.

Formula 1

ΔP∝Q^{1˜2} (Formula 1)

In the above formula, for a laminar flow, ΔP is proportional to Q while for the whole turbulent flow, ΔP is proportional to the second power of Q. In effect, it is often physically difficult to form the whole turbulent flow, though turbulent diffusion is effective for other flows than the laminar flow. This means that turbulent diffusion has a sufficient effect assuming that ΔP is proportional to the (1.5±0.4)^{th} power of Q (1.5±0.4).

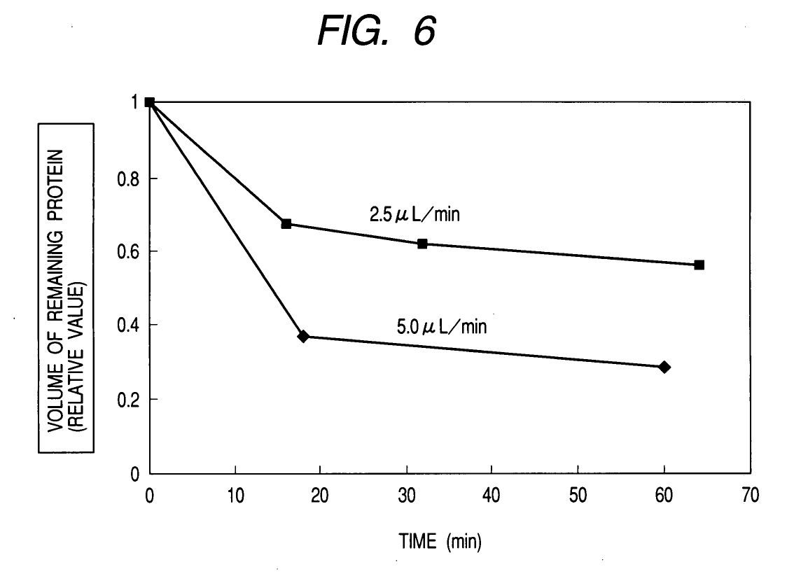

It is practical to predetermine the liquid transport condition (flow rate) so that the above-mentioned condition may be met. For example, the use of a pump for liquid chromatography enables us to examine the relationship between a liquid flow rate Q and a back pressure ΔP to the reaction element 1. This predetermination of the appropriate flow rate, which can meet the non-linear relationship in the formula shown above, achieves the chemical reaction using turbulent diffusion. FIG. 6 shows the result from enzyme-catalyzed digestion of protein. Cytochrome C protein was used for a sample material and trypsin, an enzyme, was fixed in the reaction element 1. It was verified that in the case of the flow rate being 5 μL/minute, the back pressure ΔP was proportional to the about 1.6^{th} power of Q, which met the condition shown above. In the figure, a vertical axis indicates the values (absolute) for the remaining volume of protein, while a horizontal axis indicates the reaction time. In the case of a laminar flow, the reaction should depend on the reaction time but not on the flow rate. In FIG. 6, the comparison the relationship between the cases of the flow rates 5 μL/minute and 2.5 μL/minute shows that for the later, of which flow rate is half as much as that of the former, has the same reaction time but lower reaction efficiency. This is a characteristic quality of turbulent and transient flows. It is expected that when the reaction is proceeded at a higher flow rate the reaction efficiency further improves, while the back pressure ΔP considerably increases. For that reason, attention must be paid not to cause any liquid leakage at the connections of the tubing and the capillary.

As known from the result of our experiment shown in FIG. 6, about a half of protein molecules remain in the case of the reaction time being about 15 minutes. That is because the dead volume, which is not involved in the reaction occupied about 30% of the capacity of the reaction element 1 and only one pump was used. In an actual liquid transport process, when the pump was pushed, a given flow rate can be attained but when the pump is pulled, it cannot be attained. The dead volume was reduced by a factor of two orders because the structure of the reaction element being as shown in FIG. 2A. In addition, it was verified that the use of two pumps ensure liquid transport and at a flow rate of 5 μL/minute, almost all the protein molecules were digested for 15 minutes of reaction time. Basically, assuming that the condition mentioned before is met, a sufficient effect may be attained, though attention must be paid to the dead volume in the reaction element 1 and liquid transport control.

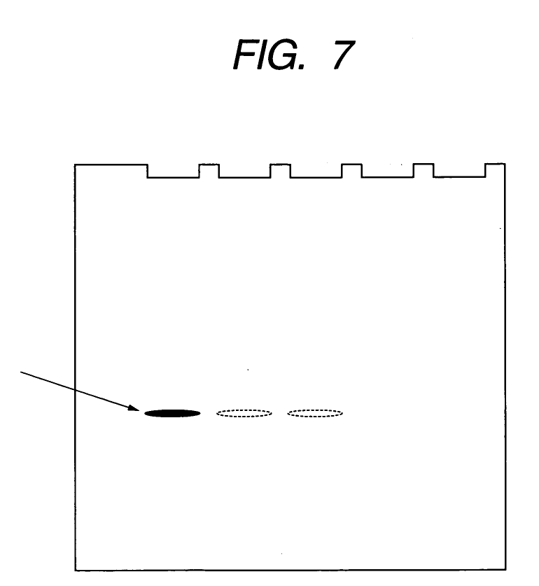

FIG. 7 shows an example of the experiment on protein digestion using two pumps in the cell having the reaction element, of which structure sis shown in FIG. 2A. In the figure, an example of an image showing separated band obtained by polyacrylamide gel electrophoresis is given. Undigested protein molecules migrated into the leftmost electrophoresis lane and the sample, to which the enzyme-catalyzed reaction was applied for 15 minutes, migrated into the lane on its right side. In the leftmost lane, the separated protein band indicated by an arrow can be observed clearly, while no bands are detected in the regions enclosed by a dotted line in the lanes on its right side. In addition, no separated protein bands were detected in the right lanes even tough the sample, to which the enzyme-catalyzed reaction was applied for 30 minute, mitigated toward them. This suggests that almost all the remaining undigested protein molecules were digested. Moreover, it is explained that peptide fragments with different masses could not be easily observed because they were produced in trace amounts by enzyme-catalyzed digestion.

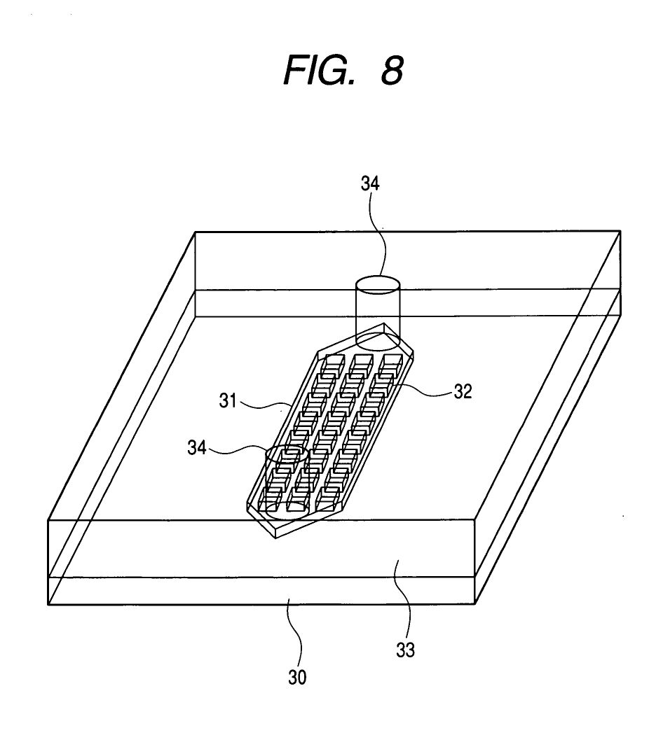

As known from an example of enzyme-fixed glass beads being arrayed almost in a line in the capillary shown in FIG. 2A, the enzyme-fixed hard micro particles (or structures) can be positioned in the flow channel to operate the reaction element under the condition mentioned before with no problem. For example, as shown in FIG. 8, the cell having a silicone substrate with a flow channel 31 and a structure 32 for causing a turbulent flow can be used with no problem. The cell can be formed by boring a hole 34 on a glass substrate 33 and jointing it to a silicone substrate 30. The micro particles (structures) for causing a turbulent flow are desirably made of any of hard materials such as glass but any materials may be used other than soft materials such as gel with no problem and resins such as PDMS (polymethyl siloxane) can be also used.

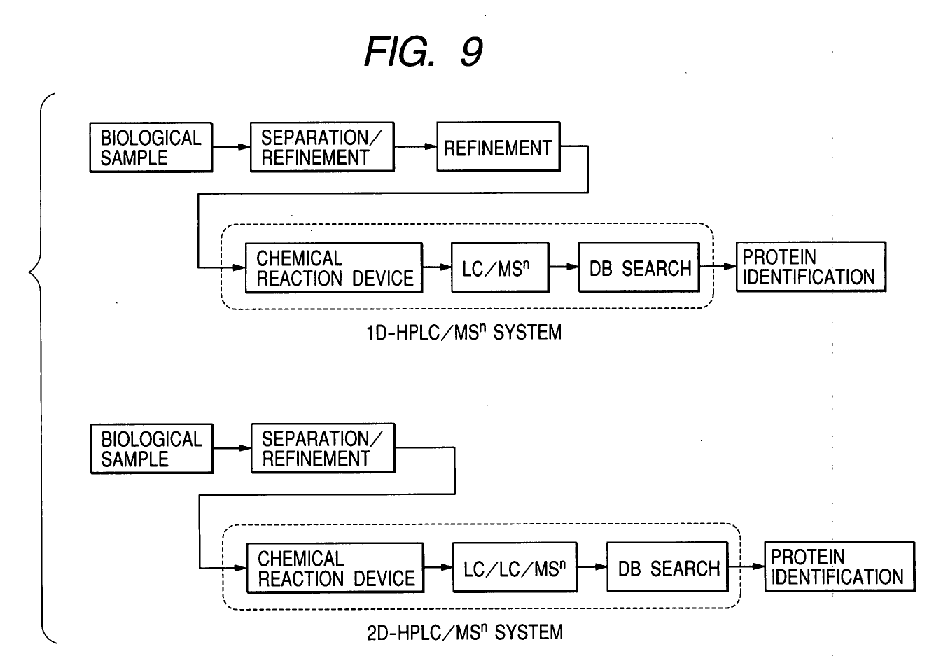

FIG. 9 shows the relationship between the structural views of the protocol for analyzing protein molecules using a liquid chromatograph mass spectrometer (LC/MS) and of an analysis system (shotgun analysis system) incorporating a chemical reaction device according to one embodiment of the present invention. In the figure, the region enclosed by a dotted line indicates the analysis system incorporating the chemical reaction device according to one embodiment of the present invention. The biological sample obtained from the biological organism is separated and refined using the liquid chromatography or affinity column method. The separated and refined sample (mixture solution containing protein molecules) undergoes peptide bond by means of a fixed digestive enzyme, for example trypsin, in the chemical reaction device. Then, The mixture solution containing peptide is separated by liquid chromatography using reverse-phase columns (1D-HPLC) or liquid chromatography using ion-exchange columns and reverse-phase columns (2D-HPLC) and the separated peptide undergoes tandem mass spectrometry (MSⁿ) in the mass analysis device. The result from tandem mass spectrometry is transferred to an information processing unit, which searches the database based on the supplied information. The result from searching identifies the protein originally existing in the sample. The conventional batch process requires 8 to 16 hours only to complete the reaction in the-chemical reaction device. Conventionally, half a day for 2D-HPLC and one hour for 1D-HPLC have been required, respectively and two days have been required if the chemical reaction process was included. In contrast, the chemical reaction device enables the result to be obtained in about half a day, improving considerably throughput. It is desired that the biological samples is obtained from the biological organisms in trace amounts as far as possible but to do so, a loss caused in the batch process must be addressed. Any loss caused through absorption by a vessel or the like is not negligible because the surface area of the sample solution increases when the sample is diluted. To solve this problem, the chemical reaction device has been designed so that the dilution of a trace amount of sample is avoided as far as possible to perform online processing, which prevents the sample from being lost, achieving higher sensitivity of the whole system. For the database for the analysis system, an internal database pre-built via data input may be used or an external database may be used to have access to update data via a server for version-up.

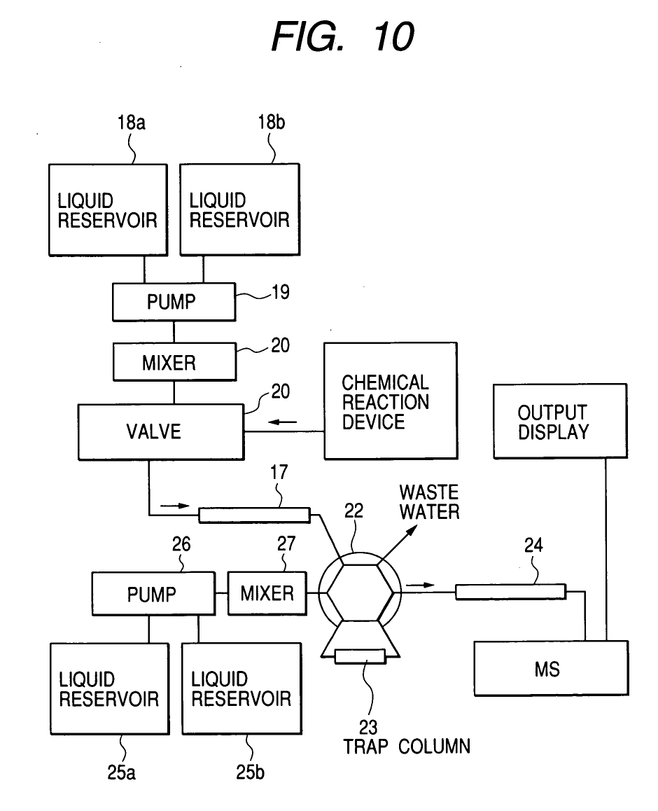

FIG. 10 shows a detail view of a 2D-HPLC element. As shown in the figure, the outlet 8 of the chemical reaction device is connected to an injection valve or a trap column of 2D-HPLC online. A solvent with a different composition is introduced into a 1D separation column 17 (for example, ion-exchange column) from a liquid reservoir 18 by a pump 19 and a mixer 20 through a valve 21. The sample separated in the 1D separation column 17 is temporarily absorbed by a trap column 23 connected to a hexagonal valve 22. Then, the solvent with a different composition is introduced into a 2D separation column 24 (for example, reverse-phase column) from a liquid reservoir 25 by a pump 26 and a mixer 27 through the valve 22. The sample separated in the 2D separation column 24 is introduced into a mass spectrometer (MS) for mass spectrometry. The output from the MS is indicated in an output display area. Similarly, in the case of 1D-HPLC being used, the chemical reaction device is connected to the system online.

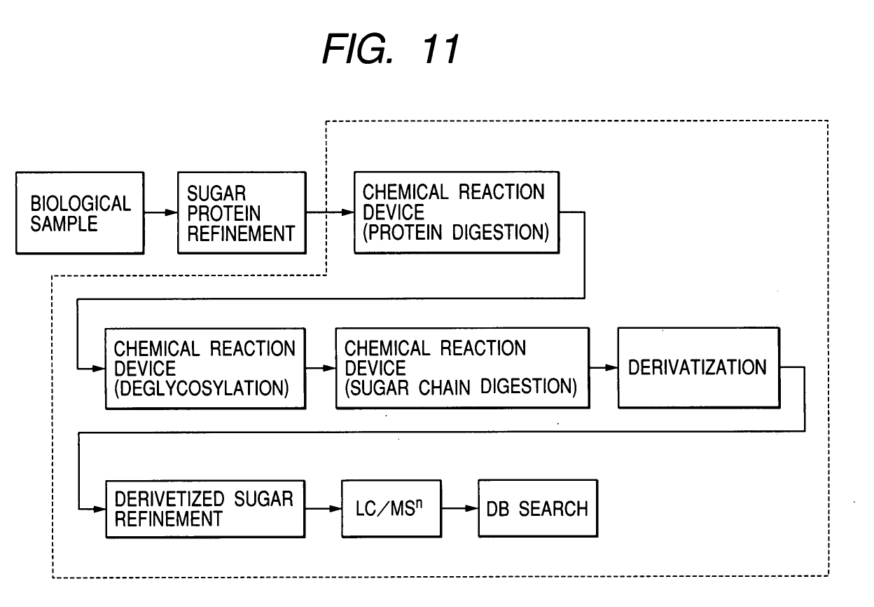

FIG. 11 shows the relationship between the structural views of the protocol for analyzing sugar chains using the mass spectrometer and of the analysis system incorporating the chemical reaction device according to one embodiment of the present invention. In the figure, the region enclosed by a dotted line indicates the analysis system according to one embodiment of the present invention. In this case, three types of chemical reactions (protein digestion, deglycosylation, and sugar chain digestion) are involved and accordingly, three types of chemical reaction devices are used as shown in the figure. Differences among them lie only in the type of enzyme to be fixed, the structure of the reaction element 1, and the reaction time and temperature of the reaction element 1. Conventionally, it has taken two days only to perform the batch chemical process but in contrast, the chemical reaction device of the preset invention can reduce the reaction time to about one hour.

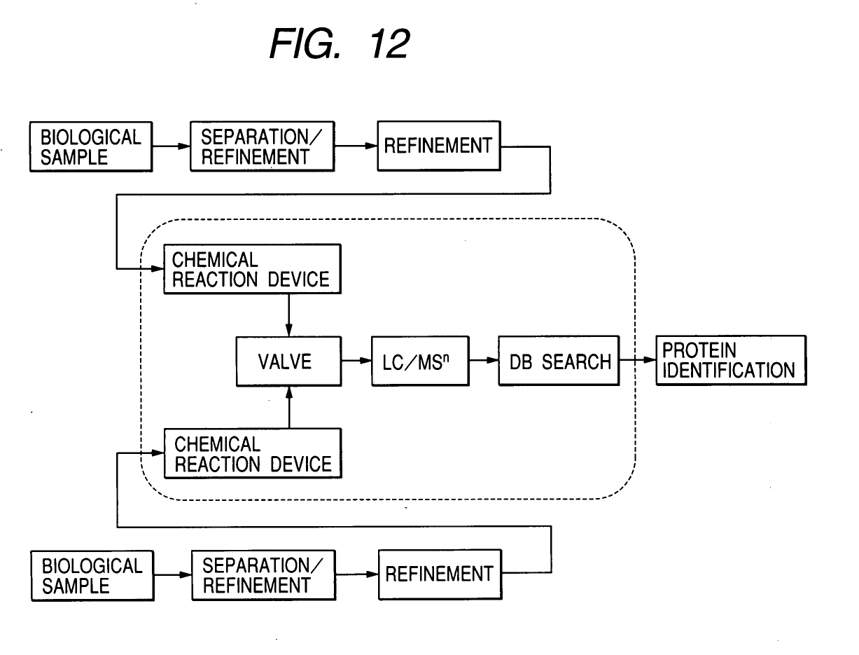

In addition, to complete liquid chromatography in a short period of time in the 1D-HPLC/MSⁿ system, a plurality of chemical reaction devices can be operated in parallel as shown in FIG. 12. In the case of this high throughput analysis,- the number of chemical reaction devices can be optimized taking the time required for sample separation by liquid chromatography into account.

The use of a chemical reaction device of the present invention ensures that a turbulent flow or a transient flow in a sample is generated in a sample flow channel containing a support, on which a chemical substance has been fixed, improving chemical reaction efficiency in the sample flow channel. In addition, the device can improve reaction efficiency between the chemical substance fixed on the support and the sample molecules in the solution by increasing the number of collisions between them. This higher reaction efficiency ensures a shorter reaction time and chemical reaction on a trace amount of biological sample with a low loss

Using the analysis system incorporating the chemical reaction device, throughput may be considerably improved because of the chemical reaction device having higher reaction efficiency. Moreover, such an ability of the analysis system that a trace amount of sample can be processed online prevents the sample from being lost, achieving higher sensitivity of the whole system.

What is claimed is:

1. A chemical reaction method comprising:
providing a sample flow channel for containing a support with biological molecules fixed on its surface,
introducing a first solution into the sample flow channel,
introducing a first air layer into the sample flow channel,
introducing a sample into the sample flow channel,
introducing a second air layer into the sample flow channel,
introducing a second solution into the sample flow channel, and
forcing the first solution, the first air layer, the sample, the second air layer, and the second solution to move so that the sample may move relative to the support, and inducing a reaction between the biological molecules and substances contained in the sample.

2. The chemical reaction method according to claim 1, wherein the biological molecules comprise an enzyme and the reaction is enzyme-catalyzed digestion of the sample and the substances contained in the sample.

3. The chemical reaction method according to claim 1 further comprising:
connecting an end of the sample flow channel to a first pipe and connecting the other end of the sample flow channel to a second pipe,
positioning the first air layer and the second air layer at the first pipe and the second pipe, respectively during the inducing the reaction.

4. The chemical reaction method according to claim 1, wherein the volume of the sample introduced when the sample is introduced is in a range, not less than 0.1 μL and not more than 100 μL.

5. The chemical reaction method according to claim 2, wherein a first volume of the introduced sample is larger than a second volume obtained by subtracting a third volume of the support from a fourth volume of internal capacity.

6. The chemical reaction method according to claim 1, wherein the sample forms a turbulent flow zone, or a transient flow zone which is a flow state between turbulent flow and laminar flow, inside the sample flow channel.

7. The chemical reaction method according to claim 1, wherein a relationship ΔP∝Q^{1.5±0.4} is established between a water pressure ΔP regulated to move the sample and a flow rate of the sample Q and the sample forms the turbulent flow zone or the transient flow zone during the inducing the reaction.

8. The chemical reaction method according to 1, further comprising:
composing the support of a plurality of micro particles and
composing the sample flow channel as a capillary.

9. The chemical reaction method according to 1, further comprising:
structuring the support to be a structure disposed at a reaction vessel and
structuring the sample flow channel as a channel disposed at the reaction vessel.

10. A chemical reaction device comprising:
a capillary for containing a plurality of micro particles and a sample,
a first pump and a second pump for controlling the flow rate of a liquid moving inside the capillary,
a first pipe, which connects to one end of the capillary and further to the first pump,
a second pipe, which connects the other end of the capillary to the second pump,
an air blowing tube, a solution feeding tube, and a sample feeding tube connected to at least either the first pipe or the second pipe, wherein
the solution feeding tube is structured to feed the solution,
the sample feeding tube is structured to feed the sample, and
the air blowing tube is structured to blow air between the fed solution and the fed sample.

11. The chemical reaction device according to claim 10, wherein the sample may reciprocate.

12. The chemical reaction device according to claim 10, further comprising a temperature regulator for regulating temperature of the capillary.

13. The chemical reaction device of claim 10, wherein the capillary is of variable length type.

14. The chemical reaction device according to claim 10, further comprising:
a connecting valve for selectively connecting the air blowing tube, the solution feeding tube, and the sample feeding tube to at least either the first pipe or the second pipe.

15. The chemical reaction device according to claim 10, further comprising:
a first connecting valve for selectively connecting the air blowing tube, the solution feeding tube, or the sample feeding tube, to the first pipe, and
a second connecting valve for selectively connecting the air blowing tube, the solution feeding tube, or the sample feeding tube to the second pipe.

16. An analysis system comprising:
a chemical reaction device comprising a capillary for containing a plurality of micro particles and a sample,
a first pump and a second pump for controlling the flow rate of a liquid moving inside the capillary,
a first pipe connected to one end of the capillary and further to the first pump,
a second pipe connected to the other end of the capillary and further to the second pump, an air blowing tube,
a solution feeding tube, and
a sample feeding tube connected to at least either the first pipe or the second pipe,
a temperature regulator for regulating temperature of the capillary,
a transport pipe for transporting the sample from the chemical reaction device,
a liquid chromatograph mass spectrometer connected to the transport pipe, and
an output display area for obtaining an output from the liquid chromatograph mass spectrometer, wherein
the solution feeding tube feeds the solution,
the sample feeding tube feeds the sample, and
the air blowing tube inserts air between the fed solution and the fed sample.

17. The analysis system according to claim 16, wherein with respect to the first pump and the second pump, a relationship ΔP∝Q^{1.5±0.4} is established between a water pressure ΔP regulated to move the sample and a flow rate of the sample Q.

18. The analysis system according to claim 16, further comprising:
a database of proteins, and
an information processing device for searching the database for protein data associated with the output from the liquid chromatograph mass spectrometer to identify proteins contained in the sample.

19. The analysis system according to claim 16, wherein a plurality of chemical reaction devices are provided, and wherein enzyme molecules are fixed to the micro particles contained in the chemical reaction devices.

20. The analysis system according to claim 16, wherein a plurality of the chemical reaction devices are provided which operate in parallel.

21. A chemical reaction method comprising:
providing a sample flow channel for containing a support with biological molecules fixed on its surface,
introducing a sample into the sample flow channel with avoiding diffusion of the sample, and
forcing the sample to move so that the sample may move relative to the support, and inducing a reaction between the biological molecules and substances contained in the sample.

22. The chemical reaction method according to claim 21 further comprising,
introducing a first solution into the sample flow channel,
introducing a first air layer into the sample flow channel,
introducing a second air layer into the sample flow channel,
introducing a second solution into the sample flow channel, and
forcing the first solution, the first air layer, the sample, the second air layer, and the second solution to move so that the sample may move relative to the support, and inducing the reaction between the biological molecules and the substances contained in the sample, wherein the first solution, the first air layer, the sample, the second air layer and the second solution are introducing into the sample flow channel in turn.
